(19) United States
(12) Patent Application Publication
(10) Pub. No.: US 2026/0241063 A1
(43) Pub. Date: Aug. 20, 2026

(54) MAGNESIUM PHOSPHATE FOR TREATMENT OF AN INFECTION

(71) Applicant: Bone Solutions, Inc., Colleyville, TX (US)

(72) Inventor: Frankie L. Morris, Colleyville, TX (US)

(21) Appl. No.: 19/542,950

(22) Filed: Feb. 18, 2026

Related U.S. Application Data
(60) Provisional application No. 63/759,598, filed on Feb. 18, 2025.

(51) Int. Cl. A61L 26/00

(52) U.S. Cl. CPC A61L 26/0004; A61L 26/0066; A61L 26/0085; A61L 2300/112; A61L 2300/232; A61L 2300/25; A61L 2300/406; A61L 2300/622; A61L 2400/18

(57) ABSTRACT

The present disclosure is directed to an antibiotic bead for wound treatment.

### CROSS-REFERENCE TO RELATED APPLICATIONS

This application claims priority to U.S. Provisional Patent Application No. 63/759,598, filed Feb. 18, 2025.

### TECHNICAL FIELD

The present disclosure relates to antibiotic beads for use in wound treatment.

### BACKGROUND

Unless otherwise indicated herein, the materials described in this section are not admitted to be prior art to the claims in this application.

Infectious wounds are commonly treated by surgical debridement of an area of tissue ischemia within the body. For instance, the standard therapy for treating chronic bone infection (osteomyelitis) includes debridement and sequestrectomy of infected bone, which is then followed by several weeks of intravenous antibiotics. Unfortunately, this treatment has several drawbacks. For example, multiple doses of antibiotics are typically needed, which can become quite expensive. In addition, the intravenous route of administration does not allow the antibiotic to be specifically directed to the location of the infection.

Intravenous administration of antibiotics also requires an operation for placement of a catheter, which can lead to serious complications. As a result, various alternatives have been proposed for providing localized treatment of bone infection. For instance, antibiotic beads have been employed that are made from bone cement (e.g., polymethylmethacrylate (PMMA)). Such beads may be selectively disposed in a void (e.g., surgical void) to locally deliver a bactericidal level of an antibiotic agent. While providing some benefits, these beads still have a variety of disadvantages. For example, most commercially available beads must be hand mixed with the antibiotic agent by a medical professional just prior to placement in a void. In addition to being an inefficient and time consuming process, such “hand-mixed” beads also tend to have an inconsistent level of the antibiotic agent and an increased risk of contamination.

As such, a need currently exists for improved antibiotic beads that are capable of being used to treat an infection in a wound site (e.g., a bone infection).

### SUMMARY

In view of the foregoing, in one embodiment of the present invention, an antibiotic bead having a size of from about 2 to about 8 millimeters is disclosed. The bead comprises one or more antibiotic agents dispersed within a magnesium phosphate and/or magnesium sulfate carrier.

In another aspect, the present disclosure provides a method of making the antibiotic bead as described herein, the method comprising
providing an aqueous solution comprising one or more antibiotic agents;
providing a dry mixture comprising MgO, magnesium sulfate, monobasic potassium phosphate, monobasic sodium phosphate, and calcium phosphate, wherein a weight percent ratio of monobasic potassium phosphate to MgO is between about 3:1 and 1:1;
mixing the aqueous solution and the dry mixture in a weight ratio of at least 5:1 to from a slurry;
optionally, adding at least one additional antibiotic agent to the slurry;
disposing the slurry in a bead mold;
allowing the slurry to set to form the antibiotic bead;
collecting the antibiotic bead from the bead mold.

In another aspect, the present disclosure provides a package comprising a plurality of the antibiotic beads as described herein.

In another aspect, the present disclosure provides a method for treating an infection, the method comprising disposing the antibiotic bead as described herein within a wound site of a patient.

In another aspect, the present disclosure provides a method for treating an infection, the method comprising disposing a plurality of the antibiotic beads as described herein within a wound site of the patient.

In another aspect, the present disclosure provides a method for treating an infection in soft tissue, the method comprising disposing a plurality of the antibiotic beads as described herein within a wound site of the patient, and allowing soft tissue to form.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

The accompanying drawings are included to provide a further understanding of the product of the disclosure, and are incorporated in and constitute a part of this specification. The drawings are not necessarily to scale, and sizes of various elements may be distorted for clarity. The drawings illustrate one or more embodiment(s) of the disclosure and together with the description serve to explain the principles and operation of the disclosure.

FIGS. 1A and 1B are X-Ray images of a dissolution of the antibiotic beads as described herein disposed in a soft tissue void.

FIGS. 2A, 2B, and 2C are images of soft tissue samples 3 weeks after use of the antibiotic bead described herein.

FIG. 3 is a graph of the elution of an antibiotic over time from the antibiotic beads as described herein.

FIG. 4 is a graph of the elution of an antibiotic over time from the two different sized antibiotic beads as described herein.

FIG. 5 are microcomputed tomography images of antibiotic beads as described herein versus a control.

### DETAILED DESCRIPTION

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The exemplary embodiments described herein are not meant to be limiting. It will be readily understood that the aspects of the present disclosure, as generally described herein and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an exemplary embodiment may include elements that are not illustrated in the Figures.

As used herein, “distal” with respect to a portion of the apparatus means the end of the device (when in use) nearer the treatment zone (e.g., the cavity in a structure) of the subject and the term “proximal” means the portion of the device (when in use) further away from the treatment zone of the subject and nearer the access site and the operator.

As used herein, with respect to measurements, “about” means +/−5%.

Generally speaking, the present invention is directed to antibiotic beads having a variety of benefits, such as inhibiting, preventing, and/or treating an infection. Typical uses of such beads may include, for instance, prophylaxis of infections for open fractures and total joint arthroplasties and in the treatment of acute and chronic osteomyelitis, periprosthetic joint infections, diabetic infections, and septic non-unions. The beads may have a variety of different cross-sectional shapes, such as generally circular, square, rectangular, ovular, elliptical, triangular, etc., as well as irregular shapes.

Regardless of their particular size and shape, the antibiotic beads comprise a magnesium phosphate and/or magnesium sulfate carrier, and in which is dispersed one or more antibiotic agents. In various embodiments as described herein, the one or more antibiotic agents are each homogenously dispersed in the magnesium phosphate and/or magnesium sulfate carrier. Through selective control over the particular nature of the matrix, the antibiotic agent(s), and the manner in which they are combined together, the present inventors have discovered that the resulting beads can be effective for treating an infection, e.g., an infection in a wound site, over a prolonged period of time. For example, such beads may be placed in a wound site, such as in a surgical site, in which bone infection has been removed, to provide the desired degree of antibiotic treatment for a time period of about 5 days or more, in some embodiments about 10 days or more, in some embodiments from about 20 days to about 60 days, and in some embodiments, from about 30 days to about 50 days (e.g., about 40 days). Without intending to be limited by theory, it is believed that the hydrophobic nature of the magnesium phosphate and/or magnesium sulfate carrier can slow the degree to which moisture diffuses into the matrix, which in turn, allows the antibiotic agent to be released gradually over the desired period of time. Also, the size and shape of the antibiotic can determine the rate of dissolution of the antibiotic agents as well.

Various embodiments of the present invention will now be described in more detail.

### I. Antibiotic Beads

The antibiotic beads as described herein may be a variety of sizes. For example, in various embodiments as described herein, the antibiotic bead has a size (e.g., diameter) of from about 1 to about 10 millimeters, or from about 2 to 8 millimeters, or from about 2 to about 6 millimeters. As used herein, the size of the bead refers to the largest diameter of the bead.

The shape of the bead is not particularly limited. For example, the beads may have a variety of different cross-sectional shapes, such as generally circular, square, rectangular, ovular, elliptical, triangular, etc., as well as irregular shapes. The antibiotic beads as described herein may also have a volume from about 0.1 to about 1 cubic centimeter per bead, in some embodiments from about 0.2 to about 0.9 cubic centimeter per bead, and in some embodiments, from about 0.3 to about 0.8 cubic centimeters per bead.

The antibiotic beads as otherwise described herein is porous. The porosity of the antibiotic bead can be determined by measuring an average normalized closed pore volume, i.e., PV/SV. To measure the average normalized closed pore volume, both the average closed pore volume (PV) and the sample volume (SV) is measured by microcomputed tomography (μCT) and the ratio is determined (PV/SV). As would be understood by the person of skill in the art, microcomputed tomography uses x-rays to make a 3D image of the internal structure of samples with micrometer resolution. For example, the average normalized closed pore volume may be measured using a Skyscan 1275 X-ray Microtomograph (Bruker). Exemplary methods of measurement by microcomputed tomography can be found in Beenken K E, Bradney L, Bellamy W, et al. 2012. “Use of xylitol to enhance the therapeutic efficacy of polymethylmethacrylate-based antibiotic therapy in treatment of chronic osteomyelitis.” Antimicrob Agents Chemother. 56:5839-44; Beenken K E, Smith J K, Skinner R A, et al. 2014. “Chitosan coating to enhance the therapeutic efficacy of calcium sulfate-based antibiotic therapy in the treatment of chronic osteomyelitis.” J Biomater Appl. 29:514-23; and Campbell M J, Beenken K E, Ramirez A M, Smeltzer M S. 2023. “The major role of sarA in limiting Staphylococcus aureus extracellular protease production in vitro is correlated with decreased virulence in diverse clinical isolates in osteomyelitis.” Virulence. 14:2175496. As would be understood by the person of skill in the art, the average normalized closed pore volume is a unitless ratio that reports the percent of a sample that contains closed pores.

In some embodiments as described herein, the antibiotic bead has an average normalized closed pore volume (i.e., PV/SV) in the range of 0.2 to 0.6%. For example, in various embodiments, the antibiotic bead has an average normalized closed pore volume (i.e., PV/SV) in the range of 0.2 to 0.55%, or in the range of 0.2 to 0.5%, or in the range of 0.2 to 0.45%. In some embodiments, the antibiotic bead has an average normalized closed pore volume (i.e., PV/SV) in the range of 0.25 to 0.6%. For example, in various embodiments, the antibiotic bead has an average normalized closed pore volume (i.e., PV/SV) in the range of 0.25 to 0.55%, or in the range of 0.25 to 0.5%, or in the range of 0.25 to 0.45%. In some embodiments, the antibiotic bead has an average normalized closed pore volume (i.e., PV/SV) in the range of 0.3 to 0.6%. For example, in various embodiments, the antibiotic bead has an average normalized closed pore volume (i.e., PV/SV) in the range of 0.3 to 0.55%, or in the range of 0.3 to 0.5%, or in the range of 0.3 to 0.45%.

In some embodiments as described herein the antibiotic bed has an average normalized closed pore volume (i.e., PV/SV) at least 1.1 times larger than a comparable bead not including the antibiotic agent. As used herein, a comparable bead is one that is the same size and shape of the antibiotic beads, made in the same way, and includes all of the same components except the antibiotic agent. For example, in some embodiments, the bead has an average normalized closed pore volume (i.e., PV/SV) at least 1.2 times, at least 1.5 time, or at least 2 times larger than a comparable bead not including the antibiotic agent.

In various embodiments as described herein, the antibiotic bead has an average normalized closed pore volume that increases when contacted with an aqueous solution (e.g., when contacted with an aqueous solution for at least 10 days). As described above, the antibiotic bead as described herein gradually releases antibiotic upon exposure to moisture. Additionally, it has been found that when compared to similar magnesium based beads but without the antibiotic agent, the porosity of the antibiotic bead is greater. It is hypothesized, without being bound by theory, that the antibiotic agent creates voids within the antibiotic bead that are exposed over time as the antibiotic agent is released. In some embodiments, the antibiotic bead has an average normalized closed pore volume that increases when contacted (e.g., for at least 10 days) with an aqueous solution by at least 1.5% (e.g. at least 1.8%, or at least 2.0%). For example, in various embodiment, the antibiotic bead has an average normalized closed pore volume that increases when contacted (e.g., for at least 10 days) with an aqueous solution in the range of 1.5%-4.5%, in the range of 1.5%-4.0%, or in the range of 1.5%-3.5%. In various embodiments, the antibiotic bead has an average normalized closed pore volume that increases when contacted (e.g., for at least 10 days) with an aqueous solution in the range of 1.8%-4.5%, in the range of 1.8%-4.0%, or in the range of 1.8%-3.5%. In various embodiments, the antibiotic bead has an average normalized closed pore volume that increases when contacted (e.g., for at least 10 days) with an aqueous solution in the range of 2.0%-4.5%, in the range of 2.0%-4.0%, or in the range of 2.0%-3.5%. In some embodiments, the antibiotic bead has an average normalized closed pore volume that increases when contacted (e.g., for at least 10 days) with an aqueous solution by at least 2.5% (e.g. at least 2.8%, or at least 3.0%, or at least 3.2%). For example, in various embodiments, the antibiotic bead has an average normalized closed pore volume that increases when contacted (e.g., for at least 10 days) with an aqueous solution in the range of 2.5%-4.5%, in the range of 2.5%-4.0%, or in the range of 2.5%-3.5%. In various embodiments, the antibiotic bead has an average normalized closed pore volume that increases when contacted (e.g., for at least 10 days) with an aqueous solution in the range of 2.8%-4.5%, in the range of 2.8%-4.0%, or in the range of 2.8%-3.5%. In various embodiments, the antibiotic bead has an average normalized closed pore volume that increases when contacted (e.g., for at least 10 days) with an aqueous solution in the range of 3.0%-4.5%, in the range of 3.0%-4.0%, or in the range of 3.0%-3.5%. In various embodiments, the antibiotic bead has an average normalized closed pore volume that increases when contacted (e.g., for at least 10 days) with an aqueous solution in the range of 3.2%-4.5%, in the range of 3.2%-4.0%, or in the range of 3.2%-3.5%.

As described above, the antibiotic beads as described herein may be beneficial in inhibiting, preventing, and/or treating an infection. Without being bound by theory, it is believed that the hydrophobic nature of the magnesium phosphate and/or magnesium sulfate carrier can slow the degree to which moisture diffuses into the matrix, which in turn, allows the antibiotic agent to be release gradually over the desired period of time. This is in contrast to otherwise identical bead comprising calcium compounds (e.g., calcium sulfate) which provide more rapid release of an antibiotic agent over time. Accordingly, in some embodiments as described herein, the antibiotic bead comprises more antibiotic agent than an antibiotic agent of an otherwise identical bead comprising a calcium compound instead of a magnesium phosphate and/or a magnesium sulfate carrier after contact with an aqueous solution for a time period of at least 5 days. As used herein, an otherwise identical bead comprising a calcium compound is a bead that is the same size and shape of the antibiotic bead as disclosed herein, is made in the same way, and includes all of the same components except it does not include a magnesium phosphate and/or a magnesium sulfate carrier. In particular, the calcium compound of the otherwise identical bead is calcium sulfate. In various embodiments as described herein, the antibiotic bead comprises more antibiotic agent than an antibiotic agent of an otherwise identical bead comprising a calcium compound instead of a magnesium phosphate and/or a magnesium sulfate carrier after contact with an aqueous solution for a time period of at least 6 day, at least 7 days, at least 8 days, or at least 9 days. For example, in various embodiments as described herein, the antibiotic bead comprises at least 20% more (e.g., at least 30% more, or at least 40% more, or at least 50% more) antibiotic agent than an antibiotic agent of an otherwise identical bead comprising a calcium compound instead of a magnesium phosphate and/or a magnesium sulfate carrier after contact with an aqueous solution for a time period of at least 5 days (e.g., at least 6 day, at least 7 days, at least 8 days, or at least 9 days). In some embodiments as described herein, the antibiotic bead comprises more antibiotic agent than an antibiotic agent of an otherwise identical bead comprising a calcium compound instead of a magnesium phosphate and/or a magnesium sulfate carrier after contact with an aqueous solution for a time period in the range of 5-9 days (e.g., in the range of 6-9 days). For example, in various embodiments, the antibiotic bead comprises at least 20% more (e.g., at least 30% more, or at least 40% more, or at least 50% more) antibiotic agent than an antibiotic agent of an otherwise identical bead comprising a calcium compound instead of a magnesium phosphate and/or a magnesium sulfate carrier after contact with an aqueous solution for a time period in the range of 5-9 days (e.g., in the range of 6-9 days).

In some embodiments as described herein, the antibiotic bead has an elution rate that is slower than an elution rate of an otherwise identical bead comprising a calcium compound instead of a magnesium phosphate and/or a magnesium sulfate carrier over a time period of 9 days of contact with an aqueous solution. For example, in some embodiments, the antibiotic bead has an elution rate that is slower than an elution rate of an otherwise identical bead comprising a calcium compound instead of a magnesium phosphate and/or a magnesium sulfate carrier over a time period of 5-9 days of contact with an aqueous solution. In various embodiments as describe herein, the antibiotic bead has an elution rate that is at least 5% (e.g., at least 10%, at least 15%, at least 20%, or at least 25%) slower than an otherwise identical bead comprising a calcium compound instead of a magnesium phosphate and/or a magnesium sulfate carrier over a time period of 9 days of contact an aqueous solution. For example, in various embodiments, the antibiotic bead has an elution rate that is at least 5% (e.g., at least 10%, at least 15%, at least 20%, or at least 25%) slower than an otherwise identical bead comprising a calcium compound instead of a magnesium phosphate and/or a magnesium sulfate carrier over a time period of 5-9 days of contact an aqueous solution.

As described herein, the antibiotic beads may be provided as a plurality, for example, in a package. In various embodiments as described herein, the plurality of antibiotic beads have a d₅₀ value of from about 1 to about 10 millimeters, or from about 2 to 8 millimeters, or from about 2 to about 6 millimeters. As used herein, the size of the bead refers to the largest diameter of the bead. As used herein, the d₅₀ particle size is the median particle size, i.e., the size of the particle at which 50% of the particles are of larger particle size and 50% are of smaller particle size. As used herein, the median particle size of a bead refers to the largest diameter of the bead.

In various embodiments as described herein, a plurality of the antibiotic beads have a narrow size distribution. For example, in various embodiments, the d₁₀ value of the plurality of antibiotic beads is at least than 50% of the d₅₀ value, e.g., at least than 70% of the d₅₀ value, or at least 85% of the d₅₀ value. In various embodiments, the doo value of the plurality of antibiotic beads is no more than 150% of the d₅₀ value, e.g., no more than 130% of the d₅₀ value, or no more than 115% of the d₅₀ value. Advantageously, in some embodiments, a plurality of antibiotic beads as described herein have a uniform size distribution. By having a uniform bead size, the amount of antibiotic and magnesium phosphate and/or magnesium sulfate carrier provided to a wound site can be easily controlled. Accordingly, in various embodiments as described herein, the d₁₀ value of the plurality of antibiotic beads is at least 95% of the d₅₀ value, e.g., at least 98% of the d₅₀ value, or at least 99% of the d₅₀ value. In various embodiments, the doo value of the plurality of antibiotic beads is no more than 105% of the d₅₀ value, e.g., no more than 102% of the d₅₀ value, or no more than 101% of the d₅₀ value. As used herein, the d₁₀ particle size is the size of the particle at which 90% of the particles are of larger particle size and 10% are of smaller particle size. As used herein, the doo particle size is the size of the particle at which 10% of the particles are of larger particle size and 90% are of smaller particle size.

A. Magnesium Phosphate and/or Magnesium Sulfate Carrier

As indicated above, the antibiotic bead comprises one or more antibiotics agents dispersed with a magnesium phosphate and/or a magnesium sulfate carrier. In some embodiments as described herein, the antibiotic bead comprises a magnesium phosphate carrier. In some embodiments as described herein, the antibiotic bead comprises a magnesium sulfate carrier.

In some embodiments as described herein, the magnesium phosphate and/or magnesium sulfate carrier contains a blend of magnesium phosphates and/or a blend of magnesium sulfates. As would be understood by the person of skill in the art, magnesium phosphate has several forms, e.g., monomagnesium phosphate, dimagnesium phosphate, and trimagnesium phosphate, and various hydrates. In some embodiments as described herein, the blend of magnesium phosphate comprises one or more of monomagnesium phosphate, dimagnesium phosphate, and trimagnesium phosphate and hydrates thereof. Similarly, as would be understood by the person of skill in the art, magnesium sulfate has several hydrate forms as well as an anhydrous form. In some embodiments as described herein, the blend of magnesium sulfates comprises one or more magnesium sulfate hydrates or anhydrous magnesium sulfate.

In some embodiments as described herein, the magnesium phosphate and/or magnesium sulfate carrier is prepared by mixing a dry mixture with an aqueous solution, wherein the dry mixture comprises MgO, magnesium sulfate, monobasic potassium phosphate, monobasic sodium phosphate, and calcium phosphate.

A suitable range for the magnesia (i.e., MgO) is generally between about 10-60 wt %, based on the weight of the dry mixture. For example, in various embodiments, MgO is present in an amount in the range of 10-50 wt %, or in the range of 20-50 wt %, or in the range of 30-50 wt %, based on the weight of the dry mixture. In some situations and/or embodiments, MgO is present in an amount in the range of 35 and 50 wt %, based on the weight of the dry mixture.

A suitable range for the magnesium sulfate is generally between about 10-60 wt %, based on the weight of the dry mixture. For example, in various embodiments, magnesium sulfate is present in an amount in the range of 10-50 wt %, or in the range of 20-50 wt %, or in the range of 30-50 wt %, based on the weight of the dry mixture. In some situations and/or embodiments, magnesium sulfate is present in an amount in the range of 35 and 50 wt %, based on the weight of the dry mixture.

In various embodiments, a suitable range for the mono potassium phosphate is generally between about 20-70 wt %, based on the weight of the dry mixture. For example, in various embodiments, mono potassium phosphate is present in an amount in the range of 20-65 wt %, or in the range of 20-60 wt %, or in the range of 20-55 wt %, or in the range of 20-50 wt %, based on the weight of the dry mixture. In various embodiments, mono potassium phosphate is present in an amount in the range of 40-70 wt %, or in the range of 40-65 wt %, or in the range of 40-60 wt %, or in the range of 40-50 wt %, based on the weight of the dry mixture.

In various embodiments, mono sodium phosphate is present in an amount in the range of 1-15 wt %, or present in an amount in the range of 1-10 wt %, based on the weight of the dry mixture. For example, in one particular example, the mono sodium phosphate is present in an amount in the range of 4-9%, based on the weight of the dry mixture. Higher percentages can be employed in certain situations.

Calcium phosphate, for example tricalcium phosphate (preferably a tricalcium apatite) and other calcium phosphates can be added in various weight percentages. The calcium containing compound(s) is/are present in an amount in the range of 1-15 wt %, or in the range of 1-10 wt %, based on the weight of the dry mixture. Higher percentages can be employed in certain situations.

In various embodiments as described herein, the weight percent ratio of monobasic potassium phosphate to MgO is between about 3:1 and 1:1.

In various embodiments as described herein, the magnesium phosphate and/or magnesium sulfate carrier comprises a majority (e.g., at least 50 wt %) of the antibiotic bead, For example, in some embodiments, the magnesium phosphate and/or magnesium sulfate carrier is present in the antibiotic bead in an amount of from about 80 wt. % to about 99 wt. %, in some embodiments from about 85 wt. % to about 98 wt. %, and in some embodiments, from about 88 wt % to about 96 wt. %, based on the total weight of the antibiotic bead.

The present inventors have determined that magnesium phosphate and/or magnesium sulfate carrier can be formed from MgO, magnesium sulfate, monobasic potassium phosphate, monobasic sodium phosphate, and calcium phosphate. As would be understood by the person or ordinary skill in the art, the amount of magnesium, potassium, calcium, and sodium can be quantified on a metallic basis regardless of the form in which these metals may be present. For example, the amount of these metals can be calculated as a weight percentage based on the total weight of metals in the antibiotic bead (i.e., on a metallic basis), i.e., without the inclusion of oxygen or non-metallic counterions in the calculation. Accordingly, in various embodiments of the disclosure as described herein, the total amount of magnesium, potassium, calcium, and sodium in the antibiotic bead is at least 80 wt %, e.g., at least 85 wt %, or at least 90 wt %, or at least 95 wt % of the antibiotic bead, on a metallic basis.

### B. Antibiotic Agent

Any of a variety of antibiotic agents may generally be employed in the beads of the present invention. Typically, it is desired that the antibiotic agent(s) employed in the beads are generally hydrophilic in nature and also effective against a broad spectrum of bacteria, such as Gram-negative bacteria (e.g., Pseudomonas, Proteus, E. coli, K. pneumoniae, Enterobacter aerogenes, Serratia, etc.) and Gram-positive (e.g., S. aureus, etc.) bacteria. In some embodiments as described herein, at least one of the one or more antibiotic agents selected is an aminoglycoside antibiotic. In some embodiments as described herein, at least one of the one or more antibiotic agents selected is an glycopeptide antibiotic. In some embodiments, the one or more antibiotic agents include at least one aminoglycoside antibiotic and at least one glycopeptide antibiotic.

Particularly suitable hydrophilic, heat-stable antibiotic agents for treating bone infection may include, for instance, vancomycin (glycopeptide), gentamycin (aminoglycoside), tobramycin (aminoglycoside), doxycycline (tetracycline), minocycline (tetracycline), etc., as well as combinations thereof. Accordingly, in some embodiments of the disclosure as described herein, the one or more antibiotic agents is selected from vancomycin, gentamycin, tobramycin, doxycycline, or minocycline. In some embodiments, the antibiotic bead comprises two or more antibiotic agents. For examples, in some embodiments, are selected from vancomycin, gentamycin, tobramycin, doxycycline, or minocycline. In some embodiments as described herein the two or more antibiotic agents are tobramycin and vancomycin.

Of course, other antibiotic agents may also be employed, such as tetracycline, amoxicillin, amoxicillin/clavulanic acid, penicillin, metronidazole, clindamycine, chlortetracycline, demeclocycline, oxytetracycline, amikacin, gentamicin, kanamycin, neomycin, netilmicin, streptomycin, tobramycin, cefadroxil, cefazolin, cephalexin, cephalothin, cephapirin, cephradine, cefaclor, cefamandole, cefametazole, cefonicid, cefotetan, cefoxitine, cefpodoxime, cefprozil, cefuroxime, cefdinir, cefixime, cefoperazone, cefotaxime, ceftazidime, ceftibuten, ceftizoxime, ceftriaxone, cefepime, azithromycin, clarithromycin, dirithromycin, erythromycin, lincomycin, troleandomycin, bacampicillin, carbenicillin, cloxacillin, dicloxacillin, meticillin, mezlocillin, nafcillin, oxacillin, piperacillin, ticarcillin, cinoxacin, ciprofloxacin, enoxacin, grepafloxacin, levofloxacin, lomefloxacin, nalidixic acid, norfloxacin, ofloxacin, sparfloxacin, sulfisoxazole, sulfacytine, sulfadiazine, sulfamethoxazole, sulfisoxazole, dapson, aztreonam, bacitracin, capreomycin, chloramphenicol, clofazimine, colistimethate, colistin, cycloserine, fosfomycin, furazolidone, methenamine, nitrofurantoin, pentamidine, rifabutin, rifampin, spectinomycin, trimethoprim, trimetrexate glucuronate, etc., as well as combinations thereof.

The dosage level of the antibiotic agents employed in the antibiotic beads may vary depending on the particular antibiotic agent employed, the size of the antibiotic bead, and the time period for which the antibiotic agent is intended to be released. Typically, however, the one or more antibiotic agents are each present in an amount in the range of from about 1 wt. % to about 20 wt. %, in some embodiments present in an amount in the range of from about 2 wt. % about 15 wt. %, and in some embodiments present in an amount in the range of from about 4 wt. % to about 12 wt. %, based on the total weight of the antibiotic bead. For example, in various embodiments as described herein, the total amount of antibiotic agent present is in an amount in the range of from about 1 wt % to about 40 wt %, or in an amount in the range of 2 wt % to about 30 wt %, or in an amount in the range of about 4 wt % to about 24 wt %, based on the total weight of the antibiotic bead.

### C. Optional Excipients

The antibiotic beads may optionally contain one or more excipients if so desired, such as radiocontrast agents, release modifiers, bulking agents, plasticizers, surfactants, crosslinking agents, flow aids, colorizing agents (e.g., chlorophyll, methylene blue, etc.), antioxidants, stabilizers, lubricants, other types of antimicrobial agents, preservatives, etc. to enhance properties and processability. When employed, the optional excipient(s) typically constitute from about 0.01 wt. % to about 20 wt. %, and in some embodiments, from about 0.05 wt. % to about 15 wt. %, and in some embodiments, from about 0.1 wt. % to about 10 wt. % of the beads. In one embodiment, for instance, a radiocontrast agent may be employed to help ensure that the beads can be detected in an X-ray based imaging technique (e.g., computed tomography, projectional radiography, fluoroscopy, etc.). Examples of such agents include, for instance, barium-based compounds, iodine-based compounds, zirconium-based compounds (e.g., zirconium dioxide), etc. One particular example of such an agent is barium sulfate. Other known antimicrobial agents and/or preservatives may also be employed to help prevent surface growth and attachment of bacteria, such as metal compounds (e.g., silver, copper, or zinc), metal salts, quaternary ammonium compounds, etc.

A release modifier may also be incorporated into the beads to help facilitate the release of the antibiotic agent(s) from the beads during use. When employed, such release modifiers(s) typically constitute from about 0.5 wt. % to about 20 wt. %, and in some embodiments, from about 1 wt. % to about 15 wt. %, and in some embodiments, from about 4 wt. % to about 10 wt. % of the beads.

One example of such a release modifier is a hydrophilic polymer that is soluble and/or swellable in water. Examples of such hydrophilic polymers include, for instance, alginates (e.g., sodium, potassium and calcium alginates), carboxymethylcellulose, agar, gelatin, polyvinyl alcohols, polyalkylene glycols, collagen, pectin, chitin, chitosan, poly-1-caprolactone, polyvinylpyrrolidone, polysaccharides, hydrophilic polyurethane, polyhydroxyacrylate, dextran, xanthan, hydroxypropyl cellulose, methyl cellulose, and homopolymers and copolymers of N-vinylpyrrolidone, N-vinyllactam, N-vinyl butyrolactam, N-vinyl caprolactam, other vinyl compounds having polar pendant groups, acrylate and methacrylate having hydrophilic esterifying groups, hydroxyacrylate, acrylic acid, and combinations thereof. Particularly suitable hydrophilic polymers are polyalkylene glycols, such as those having a molecular weight of from about 100 to 500,000 grams per mole, in some embodiments from about 500 to 200,000 grams per mole, and in some embodiments, from about 1,000 to about 100,000 grams per mole. Specific examples of such polyalkylene glycols include, for instance, polyethylene glycols, polypropylene glycols polytetramethylene glycols, polyepichlorohydrins, etc.

As described above, the antibiotic beads of the disclosure is mostly composed of the magnesium phosphate and/or magnesium sulfate carrier formed from MgO, magnesium sulfate, monobasic potassium phosphate, monobasic sodium phosphate, and calcium phosphate. In some embodiments as described herein, the antibiotic bead has an additional metal of no more than 10 wt % based on the total weight of the bead. The additional metal may be any metal that is not magnesium, potassium, calcium, or sodium. For example, in various embodiments, the antibiotic bead has an additional metal content of no more than 5 wt %, or no more than 2 wt %, or no more than 1 wt %, based on the total weight of the bead.

In some embodiments as described herein, the antibiotic bead has a calcium sulfate content of no more than 10 wt %, based on the total weight of the bead. For example, in various embodiments, the antibiotic bead has a calcium sulfate content of no more than 5 wt %, or no more than 2 wt %, or no more than 1 wt %, based on the total weight of the bead. In some embodiments as described herein, the antibiotic bead comprises substantially no calcium sulfate. For example, in various embodiments, the antibiotic bead comprises no more than 0.5 wt %, or no more than 0.2 wt %, or no more than 0.1 wt % calcium sulfate, based on the total weight of the bead, on a metallic basis.

### II. Methods of Making Antibiotic Beads

In another aspect, the present disclosure provides a method for making the antibiotic bead as described herein. The method includes providing an aqueous solution comprising one or more antibiotic agents; providing a dry mixture comprising MgO, magnesium sulfate, monobasic potassium phosphate, monobasic sodium phosphate, and calcium phosphate, wherein a weight percent ratio of monobasic potassium phosphate to MgO is between about 3:1 and 1:1; mixing the aqueous solution and the dry mixture in a weight ratio of at least 5:1 to from a slurry; optionally, adding at least one additional antibiotic agent to the slurry; disposing the slurry in a bead mold; allowing the slurry to set to form the antibiotic bead; collecting the antibiotic bead from the bead mold.

In some embodiments as described herein, the weight ratio of the aqueous solution and the dry mixture is at least 4:1, e.g., at least 3:1. For example, in various embodiments, the weight ratio of the aqueous solution and the dry mixture is in the range of 5:1 to 2:1, e.g., in the range of 4:1 to 2:1, or in the range of 5:1 to 3:1, or in the range of 4:1 to 3:1.

As described above, the aqueous solution includes the one or more antibiotic agents. The one or more antibiotic agents may be selected from the antibiotic agents as otherwise described herein. For example, in some embodiments, the one or more antibiotic agents is selected from vancomycin, gentamycin, tobramycin, doxycycline, or minocycline. As would be understood by the person of ordinary skill in the art, the one or more antibiotic agents are soluble in aqueous solution and provided to the dry mixture as a liquid. For example, one or more antibiotic agents may be soluble in water (e.g., sterile water or other sterile aqueous solution, e.g., slight saline solution).

Optionally, the method includes adding at least one additional antibiotic agent to the slurry. In this case, such an additional antibiotic agent may be added as a solid. The at least one additional antibiotic agent may be selected from the antibiotic agents as otherwise described herein. For example, in some embodiments, the at least one additional antibiotic agents is selected from vancomycin, gentamycin, tobramycin, doxycycline, or minocycline.

As described above, the method includes disposing the slurry in a bead mold and allowing the slurry to set to form the antibiotic bead. Various bead molds may be used. In particular embodiments, the bead mold as described in US Design patent application Ser. No. 29/936,528, filed Apr. 9, 2024, which is hereby incorporated herein by reference in its entirety, is used. The slurry may be allowed to set for a sufficient time (i.e., a setting time) and at a sufficient temperature (i.e., a setting temperature) in the bead mold. For example, in some embodiments, the setting time is at least 10 minutes, or at least 20 minutes, or at least 30 minutes. In some embodiments, the setting temperature is ambient or room temperature.

After the slurry is set to form the antibiotic bead, the antibiotic bead is collected from the bead mold to provide the antibiotic bead of the disclosure.

### III. Packages

Another aspect of the present disclosure provides a package comprising a plurality of antibiotic beads having a size of from about 2 to about 8 millimeters, wherein the beads comprise one or more antibiotic agents dispersed within a magnesium phosphate and/or a magnesium sulfate carrier. The antibiotic beads are as otherwise described herein.

The package as otherwise described herein includes a plurality of antibiotic beads and the number of beads is not particularly limited. In some embodiments as described herein, the package includes a number of beads to provide an effective dose of antibiotic agents in one package. For example, in various embodiments, the package contains from 2 to 1600 antibiotic beads.

In some embodiments, the package may include antibiotic beads that include at least one different antibiotic agent. In some embodiments, the package may include one or more antibiotic beads, as otherwise described herein, each including one or more different antibiotics. For example, in some embodiments, the package contains a plurality of first beads comprising a first antibiotic agent and a plurality of second beads comprising a second antibiotic agent, wherein the first antibiotic agent is different than the second antibiotic agent. In some embodiments as described herein, the package contains a plurality of first beads comprising an antibiotic agent at a first concentration and a plurality of second beads comprising an antibiotic agent at a second concentration, wherein the second concentration is greater than the first concentration.

### IV. Methods of Use

Another aspect of the present disclosure provides a method for treating an infection, the method comprising disposing the antibiotic bead as described herein within a wound site of a patient. In some embodiments, the antibiotic bead is allowed to remain within the wound site for a time period in the range of about 5 to 90 days or more (e.g., of about 5 to 70 days, about 5 to 50 day, about 5 to 20 days, or about 5 t0 10 days). For example, in some embodiments, the antibiotic bead is allowed to remain within the wound site for a time period in the range of about 10 to 90 days or more (e.g., of about 10 to 70 days, about 10 to 50 day, or about 10 to 20 days). In some embodiments, the antibiotic bead is allowed to remain within the wound site for a time period in the range of about 20 to 90 days or more (e.g., of about 20 to 70 days, or about 20 to 50 days). In some embodiments as described herein the beads are allowed to remain within the wound site for a time period in the range of about 20 to 90 days or more. In some embodiments, the wound site includes a void in which infectious bone has been surgically removed, and wherein the bead is disposed within the void.

Another aspect of the present disclosure provides a method for treating an infection, the method comprising disposing a plurality of beads within a wound site of a patient that have a size of from about 2 to about 8 millimeters, wherein the beads comprise one or more antibiotic agents dispersed within a magnesium phosphate and/or a magnesium sulfate carrier. The plurality of antibiotic beads may be the beads as described herein of provided from the package as described herein. In some embodiments, the antibiotic bead is allowed to remain within the wound site for a time period in the range of about 5 to 90 days or more (e.g., of about 5 to 70 days, about 5 to 50 day, about 5 to 20 days, or about 5 t0 10 days). For example, in some embodiments, the antibiotic bead is allowed to remain within the wound site for a time period in the range of about 10 to 90 days or more (e.g., of about 10 to 70 days, about 10 to 50 day, or about 10 to 20 days). In some embodiments, the antibiotic bead is allowed to remain within the wound site for a time period in the range of about 20 to 90 days or more (e.g., of about 20 to 70 days, or about 20 to 50 days). In some embodiments as described herein the beads are allowed to remain within the wound site for a time period in the range of about 20 to 90 days or more. In some embodiments as described herein the beads are allowed to remain within the wound site for a time period in the range of about 20 to 90 days or more. In some embodiments, the wound site includes a void in which infectious bone has been surgically removed, and wherein the bead is disposed within the void.

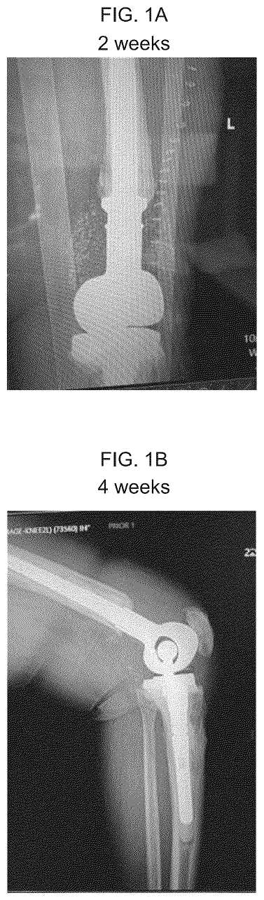

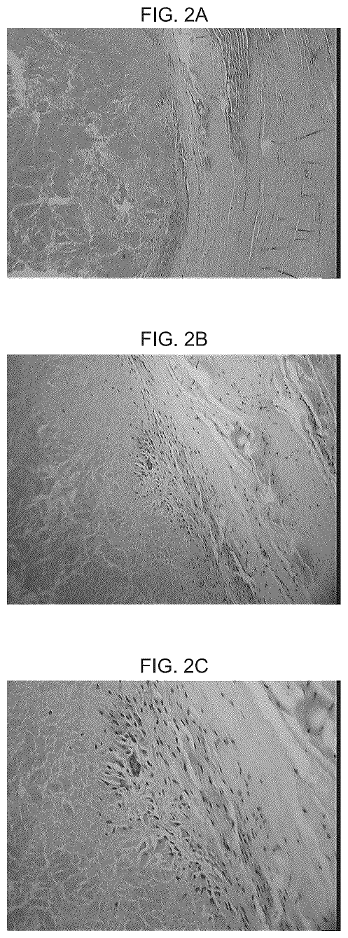

Another aspect of the present disclosure provides a method for treating an infection in soft tissue, the method comprising disposing a plurality of antibiotic beads within a wound site of a patient that have a size of from about 2 to about 8 millimeters, wherein the antibiotic beads comprise one or more antibiotic agents dispersed within a magnesium phosphate and/or a magnesium sulfate carrier; and allowing soft tissue to form. For example, in various embodiments, the plurality of antibiotic beads may be as otherwise described herein. FIGS. 1A and 1B are X-Ray images of a dissolution of the antibiotic beads as described herein disposed in a soft tissue void after 2 weeks and after 4 weeks, respectively. FIGS. 2A, 2B, and 2C are images of soft tissue samples 3 weeks after use of the antibiotic bead described herein. From these figures, less inflammation is observed in the tissue.

### IV. Examples

The Examples that follow are illustrative of specific embodiments of the process of the disclosure, and various uses thereof. They are set forth for explanatory purposes only, and are not to be taken as limiting the scope of the disclosure.

### Example 1. Preparation of Antibiotic Beads

Vancomycin was obtained from the inpatient pharmacy at the University of Arkansas for Medical Sciences. A control bone fill kits was obtained from Stimulan (CaSO₄ hemihydrate control beads). A dry mixture of MgO, magnesium sulfate, monobasic potassium phosphate, monobasic sodium phosphate, and calcium phosphate, as otherwise described herein was prepared. 1.0 μm of vancomycin powder was added to the 1.2 ml liquid component of each kit. After mixing, the liquid component containing vancomycin was added to the powder component. After 20 min, 3 mm magnesium-based antibiotic beads of the present disclosure and calcium-based control beads were formed using the template. Control beads were made without vancomycin to determine whether the addition of vancomycin impacted set time and structural integrity.

To assess antibiotic elution, 100 (2 mm) or 50 (3 mm) beads were weighed and placed in individual wells of a 6-well microtiter plate containing 2 mL of phosphate buffered saline (PBS). Plates were incubated at 37° C. on an orbital shaker to ensure continuous mixing. After 1 hr, the buffer was removed in its entirety and replaced with 2.0 ml of fresh PBS. Analysis of antibiotic activity in this eluate was defined as the amount of vancomycin observed on day 0 (DO). Every 24 hours for 9 days the buffer was removed in its entirety and replaced with 2 ml of fresh PBS.

### Example 2. Antibiotic Elution

100 μL of eluate was added to 100 μl of 2× tryptic soy broth (TSB) in a 96-well microtiter plate before making 2-fold dilutions in 1×TSB. Using the CC8, USA300 strain LAC, 105 colony-forming units were then added to each well. Plates were incubated overnight at 37° C. to determine the highest 2-fold dilution that contained enough vancomycin to prevent bacterial growth. To approximate the amount of antibiotic in this well, results were compared to the activity observed with a known concentration of freshly prepared vancomycin. Given the use of 2-fold dilutions, this assay yields a 2-fold range of active vancomycin; results are reported as the lower value in this range.

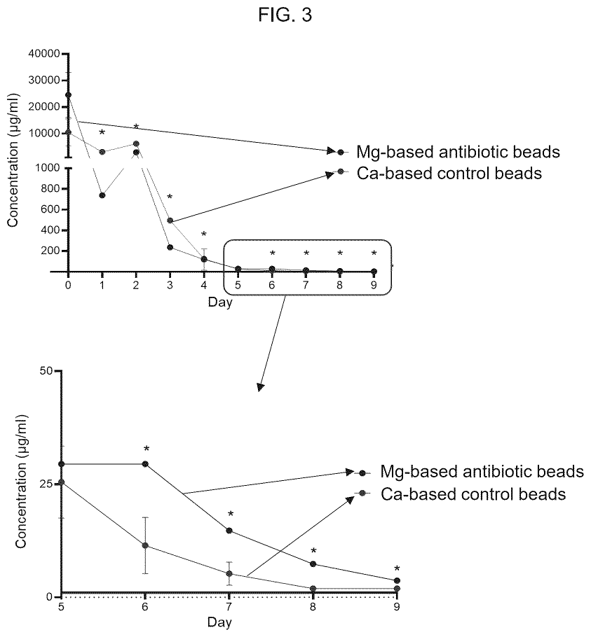

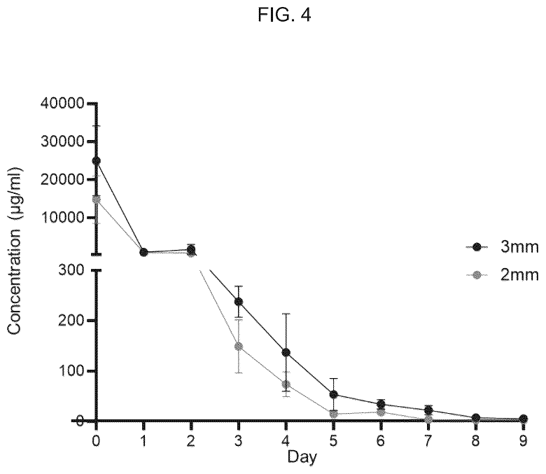

All assays were done in triplicate. Results are reported as the average amount of active vancomycin in each sample after standardization by weight. Because bead size also impacts bead volume and surface area, the same protocol was used to compare antibiotic elution from 2 and 3 mm antibiotic beads as otherwise described herein. Statistical analysis was done by students T-test comparing the amount of vancomycin in the eluate at each time point (D0-D9). Comparisons were made using GraphPad Prism (version 10.0.0 for Windows, GraphPad Software) with a p-value≤0.05 considered statistically significant. FIG. 3 is a graph of the elution of vancomycin over time from the magnesium-based antibiotic beads as described herein versus the calcium-based control beads. FIG. 4 is a graph of the elution of vancomycin over time from the two different sized antibiotic beads as described herein.

When loaded with equal amounts of vancomycin, the CaSO4 hemihydrate control beads and antibiotic beads as otherwise described herein had similar overall patterns of in vitro elution. After normalizing by weight, the average amount of vancomycin eluted from 3 mm antibiotic beads as otherwise described herein on DO was 24,560 μg/mL per gram, while the maximum amount observed with 3 mm CaSO4 control beads was 10,453 μg/mL per gram. However, the difference between the two groups was not significant, likely owing to the high concentrations observed at this time point and the limitation imposed by the use of 2-fold dilutions. By D5 vancomycin concentrations had fallen to <30 μg/ml per gram and were comparable between the antibiotic beads as otherwise described herein and the control beads. However, on days 6 through 9 the concentration observed with antibiotic as otherwise described herein was significantly higher than that observed with control beads.

Differences in bead volume and surface area can also impact antibiotic. Thus, a 2nd experiment was carried out in which vancomycin elution was assessed for 2 vs. 3 mm antibiotic beads of the present disclosure. The overall elution profiles were similar, but after adjusting for weight the amount of vancomycin was higher with 3 than 2 mm beads, although the differences observed on any given day were not statistically significant. The results observed with 3 mm antibiotic beads in this 2nd experiment were consistent with those observed with 3 mm beads in the 1st experiment, thus illustrating the reproducibility of the experimental protocol.

### Example 3. Characterization of Antibiotic Beads

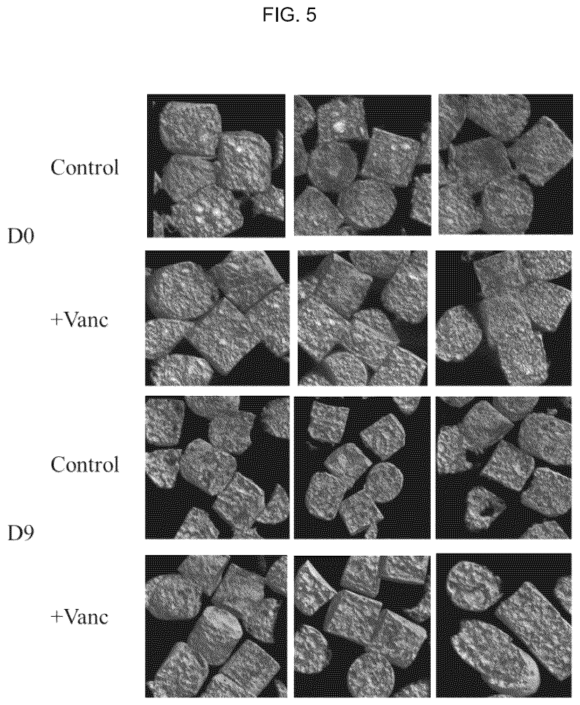

The impact of vancomycin on the structural integrity of the antibiotic beads was assessed by μCT on DO and D9. Image acquisition was done as previously described (20, 22, 24). Briefly, a Skyscan 1275 X-ray Microtomograph (Bruker) was used with an isotropic voxel size of 26.3 μm and an X-ray voltage of 40 kV (100 μA). Reconstruction was carried out using the Skyscan Nrecon software and scans were cropped to the area containing the samples using Skyscan Data Viewer. Images were then generated in Skyscan CTvox and are shown in FIG. 5.

For analysis, the reconstructed cross-sectional slices were processed using the Skyscan CT-analyzer software to perform a semi-automated protocol to create preliminary regions of interest (ROIs). The semi-automated protocol was then used to focus on the beads as much as possible by thresholding from 50-255, de-speckling, eroding, and dilating the ROIs. The resulting ROIs were loaded and corrected manually, then a threshold of 50-255 was again set in CT-analyzer and the volumes and porosity at each time point determined by calculating the pore volume as a function of sample volume (PV/SV) with results reported as the change in PV/SV over time (ΔPV/SV). Statistical analysis was done by students T-tests with a p-value≤0.05 considered statistically significant. The results are shown in Table 1, where PV represents the closed pore volume and SV represents the sample volume, so PV/SV is the closed pore volume normalized by sample volume. ΔPV/SV is the change in the PV/SV from D0 to D10. * and ** both indicate p-value <0.01 between the ΔPV/SV of those groups via students t-test.

TABLE 1

| | | D0 | D10 | |
|---|---|---|---|---|
| | sample | PV/SV | PV/SV | ΔPV/SV |
| 3 mm + Vancomycin*^{,} ** | 1 | 0.23% | 3.69% | 3.46% |
| | 2 | 0.42% | 3.77% | 3.35% |
| | 3 | 0.50% | 4.17% | 3.67% |
| | avg | 0.38% | 3.88% | 3.49% |
| 2 mm + Vancomycin* | 4 | 0.25% | 2.08% | 1.83% |
| | 5 | 0.41% | 3.03% | 2.61% |
| | 6 | 0.56% | 2.45% | 1.90% |
| | avg | 0.41% | 2.52% | 2.11% |
| 3 mm Control** | 7 | 0.10% | 2.16% | 2.06% |
| | 8 | 0.19% | 2.40% | 2.21% |
| | 9 | 0.19% | 2.37% | 2.18% |
| | avg | 0.16% | 2.31% | 2.15% |
| 2 mm Control | 10 | 0.34% | 2.67% | 2.33% |
| | 11 | 0.22% | 3.06% | 2.85% |
| | 12 | 0.53% | 2.57% | 2.04% |
| | avg | 0.36% | 2.77% | 2.40% |

To assess the impact of vancomycin on the structural integrity of the antibiotic beads, μCT was used to visualize 3 mm beads with and without vancomycin on D0 and D9. There was no visual evidence that the addition of vancomycin altered the stability or structural integrity of the antibiotic beads. However, the ratio of closed pore volume (PV) to sample volume (SV) increased from D0 to D9 (p≤0.001), and the change in PV/SV (ΔPV/SV) was greater in beads with vancomycin than in beads without vancomycin (p=0.0002). This was true irrespective of bead size (2 mm vs. 3 mm). The addition of vancomycin did not impact the set time of the antibiotic beads irrespective of bead size.

The results reported here demonstrate that the levels of vancomycin in the eluate were comparable between the antibiotic beads and the control beads during the early time period, but that prolonged elution was extended to a statistically significant extent with the antibiotic beads. To the extent that both the maximum and prolonged levels of antibiotic provide a therapeutic benefit, this suggests that the antibiotic beads is comparable to control beads and may even be superior, particularly with respect to prolonged elution. Moreover, setting time was not different between antibiotic loaded and non-antibiotic loaded magnesium based beads, and porosity was increased, which might suggest the potential for decreased resorption time. The increase in porosity suggests that the addition of vancomycin creates voids within the antibiotic beads that are exposed over time as the antibiotic is released. Definitively assessing the therapeutic relevance of these observations will require in vivo studies, as will the possibility that adding vancomycin and other classes of antibiotic will impact bone regeneration and repair. Nevertheless, the results provide justification for the use of the antibiotic beads for local delivery of antibiotics to address therapeutic efficacy and bone repair.

Various aspects and embodiments of the disclosure are provided by the following enumerated embodiments, which may be combined in any number and in any fashion not logically or technically inconsistent.
Embodiment 1. An antibiotic bead having a size of from about 2 to about 8 millimeters, wherein the antibiotic bead comprises:
   one or more antibiotic agents dispersed within a magnesium phosphate and/or a magnesium sulfate carrier.
Embodiment 2. The antibiotic bead of embodiment 1, wherein the bead comprises a magnesium phosphate carrier.
Embodiment 3. The antibiotic bead of embodiment 1 or embodiment 2, wherein the bead comprises a magnesium sulfate carrier.
Embodiment 4. The antibiotic bead of any of embodiments 1-3, wherein the magnesium phosphate and/or magnesium sulfate carrier is prepared by mixing a dry mixture with an aqueous solution, wherein the dry mixture comprises MgO, magnesium sulfate, monobasic potassium phosphate, monobasic sodium phosphate, and calcium phosphate.
Embodiment 5. The antibiotic bead of embodiment 4, wherein a weight percent ratio of monobasic potassium phosphate to MgO is between about 3:1 and 1:1.
Embodiment 6. The antibiotic bead of embodiment 1, wherein the bead has a generally circular cross-sectional shape.
Embodiment 7. The antibiotic bead of embodiment 1, wherein the bead has a generally hexagon cross-sectional shape.
Embodiment 8. The antibiotic bead of embodiment 1, wherein the bead has a generally square cross-sectional shape.
Embodiment 9. The antibiotic bead of any of embodiments 1-8, wherein the bead has an average normalized closed pore volume (i.e., PV/SV) in the range of 0.2 to 0.6% (e.g., in the range of 0.2 to 0.55%, or 0.2 to 0.5%, or 0.2 to 0.45%).
Embodiment 10. The antibiotic bead of any of embodiments 1-8, wherein the bead has an average normalized closed pore volume (i.e., PV/SV) in the range of 0.25 to 0.6% (e.g., in the range of 0.25 to 0.55%, or 0.25 to 0.5%, or 0.25 to 0.45%).
Embodiment 11. The antibiotic bead of any of embodiments 1-8, wherein the bead has an average normalized closed pore volume (i.e., PV/SV) in the range of 0.3 to 0.6% (e.g., in the range of 0.3 to 0.55%, or 0.3 to 0.5%, or 0.3 to 0.45%).
Embodiment 12. The antibiotic bead of any of embodiments 1-11, wherein the bead has an average normalized closed pore volume (i.e., PV/SV) at least 1.1 times larger than a comparable bead not including the antibiotic agent.
Embodiment 13. The antibiotic bead of any of embodiments 1-11, wherein the bead has an average normalized closed pore volume (i.e., PV/SV) at least 1.2 times larger than a comparable bead not including the antibiotic agent.
Embodiment 14. The antibiotic bead of any of embodiments 1-11, wherein the bead has an average normalized closed pore volume (i.e., PV/SV) at least 1.5 times larger than a comparable bead not including the antibiotic agent.
Embodiment 15. The antibiotic bead of any of embodiments 1-11, wherein the bead has an average normalized closed pore volume (i.e., PV/SV) at least 2 times larger than a comparable bead not including the antibiotic agent.
Embodiment 16. The antibiotic bead of any of embodiments 1-15, wherein the bead has an average normalized closed pore volume that increases when contacted with an aqueous solution.
Embodiment 17. The antibiotic bead of any of embodiments 1-15, wherein the bead has an average normalized closed pore volume that increases when contacted with an aqueous solution by at least 1.5% (e.g. at least 1.8%, or at least 2.0%).
Embodiment 18. The antibiotic bead of any of embodiments 1-15, wherein the bead has an average normalized closed pore volume that increases when contacted with an aqueous solution in the range of 1.5%-4.5% (e.g. in the range of 1.5%-4.0%, or 1.5%-3.5%).
Embodiment 19. The antibiotic bead of any of embodiments 1-15, wherein the bead has an average normalized closed pore volume that increases when contacted with an aqueous solution in the range of 1.8%-4.5% (e.g. in the range of 1.8%-4.0%, or 1.8%-3.5%).
Embodiment 20. The antibiotic bead of any of embodiments 1-15, wherein the bead has an average normalized closed pore volume that increases when contacted with an aqueous solution in the range of 2.0%-4.5% (e.g. in the range of 2.0%-4.0%, or 2.0%-3.5%).
Embodiment 21. The antibiotic bead of any of embodiments 1-15, wherein the bead has an average normalized closed pore volume that increases when contacted with an aqueous solution by at least 2.5% (e.g. at least 2.8%, or at least 3.0%, or at least 3.2%).
Embodiment 22. The antibiotic bead of any of embodiments 1-15, wherein the bead has an average normalized closed pore volume that increases when contacted with an aqueous solution in the range of 2.5%-4.5% (e.g. in the range of 2.5%-4.0%, or 2.5%-3.5%).
Embodiment 23. The antibiotic bead of any of embodiments 1-15, wherein the bead has an average normalized closed pore volume that increases when contacted with an aqueous solution in the range of 2.8%-4.5% (e.g. in the range of 2.8%-4.0%, or 2.8%-3.5%).
Embodiment 24. The antibiotic bead of any of embodiments 1-15, wherein the bead has an average normalized closed pore volume that increases when contacted with an aqueous solution in the range of 3.0%-4.5% (e.g. in the range of 3.0%-4.0%, or 3.0%-3.5%).
Embodiment 25. The antibiotic bead of any of embodiments 1-15, wherein the bead has an average normalized closed pore volume that increases when contacted with an aqueous solution in the range of 3.2%-4.5% (e.g. in the range of 3.2%-4.0%, or 3.2%-3.5%).
Embodiment 26. The antibiotic bead of any of embodiments 1-25, wherein the bead comprises more antibiotic agent than an antibiotic agent of an otherwise identical bead comprising a calcium compound instead of a magnesium phosphate and/or a magnesium sulfate carrier after contact with an aqueous solution for a time period of at least 5 days (e.g., at least 6 day, at least 7 days, at least 8 days, or at least 9 days).
Embodiment 27. The antibiotic bead of any of embodiments 1-26, wherein the bead comprises at least 20% more (e.g., at least 30% more, or at least 40% more, or at least 50% more) antibiotic agent than an antibiotic agent of an otherwise identical bead comprising a calcium compound instead of a magnesium phosphate and/or a magnesium sulfate carrier after contact with an aqueous solution for a time period of at least 5 days (e.g., at least 6 day, at least 7 days, at least 8 days, or at least 9 days).
Embodiment 28. The antibiotic bead of any of embodiments 1-27, wherein the bead comprises more antibiotic agent than an antibiotic agent of an otherwise identical bead comprising a calcium compound instead of a magnesium phosphate and/or a magnesium sulfate carrier after contact with an aqueous solution for a time period in the range of 5-9 days (e.g., in the range of 6-9 days).
Embodiment 29. The antibiotic bead of any of embodiments 1-28, wherein the bead comprises at least 20% more (e.g., at least 30% more, or at least 40% more, or at least 50% more) antibiotic agent than an antibiotic agent of an otherwise identical bead comprising a calcium compound instead of a magnesium phosphate and/or a magnesium sulfate carrier after contact with an aqueous solution for a time period in the range of 5-9 days (e.g., in the range of 6-9 days).
Embodiment 30. The antibiotic bead of any of embodiments 1-29, wherein the bead has an elution rate that is slower than an elution rate of an otherwise identical bead comprising a calcium compound instead of a magnesium phosphate and/or a magnesium sulfate carrier over a time period of 9 days of contact with an aqueous solution.
Embodiment 31. The antibiotic bead of any of embodiments 1-30, wherein the bead has an elution rate that is slower than an elution rate of an otherwise identical bead comprising a calcium compound instead of a magnesium phosphate and/or a magnesium sulfate carrier over a time period of 5-9 days of contact with an aqueous solution.
Embodiment 32. The antibiotic bead of any of embodiments 1-31, wherein the bead has an elution rate that is at least 5% (e.g., at least 10%, at least 15%, at least 20%, or at least 25%) slower than an otherwise identical bead comprising a calcium compound instead of a magnesium phosphate and/or a magnesium sulfate carrier over a time period of 9 days of contact an aqueous solution.
Embodiment 33. The antibiotic bead of any of embodiments 1-32, wherein the bead has an elution rate that is at least 5% (e.g., at least 10%, at least 15%, at least 20%, or at least 25%) slower than an otherwise identical bead comprising a calcium compound instead of a magnesium phosphate and/or a magnesium sulfate carrier over a time period of 5-9 days of contact an aqueous solution.
Embodiment 34. The antibiotic bead of any of embodiments 1-33, wherein at least one of the one or more antibiotic agents selected is an aminoglycoside antibiotic.
Embodiment 35. The antibiotic bead of any of embodiments 1-34, wherein at least one of the one or more antibiotic agents selected is an glycopeptide antibiotic.
Embodiment 36. The antibiotic bead of any of embodiments 1-35, wherein the one or more antibiotic agents is selected from vancomycin, gentamycin, tobramycin, doxycycline, or minocycline.
Embodiment 37. The antibiotic bead of any of embodiments 1-35, comprising two or more antibiotic agents.
Embodiment 38. The antibiotic bead of embodiment 37, wherein the two or more antibiotic agents are selected from vancomycin, gentamycin, tobramycin, doxycycline, or minocycline.
Embodiment 39. The antibiotic bead of any of embodiments 1-38, wherein the bead further comprises a release modifier, a radiocontrast agent, a colorizing agent, an antimicrobial agent and/or preservative.
Embodiment 40. The antibiotic bead of any of embodiments 1-39, having a calcium sulfate content of no more than 5 wt % (e.g., no more than 2 wt %, or no more than 1 wt %), based on the total weight of the bead.
Embodiment 41. The antibiotic bead of any of embodiments 1-40, comprising substantially no (e.g., no more than 0.5 wt %, or no more than 0.2 wt %, or no more than 0.1 wt %) calcium sulfate.
Embodiment 42. A method of making the antibiotic bead of any of embodiments 1-41, the method comprising
   providing an aqueous solution comprising one or more antibiotic agents;
   providing a dry mixture comprising MgO, magnesium sulfate, monobasic potassium phosphate, monobasic sodium phosphate, and calcium phosphate, wherein a weight percent ratio of monobasic potassium phosphate to MgO is between about 3:1 and 1:1;
   mixing the aqueous solution and the dry mixture in a weight ratio of at least 5:1 to from a slurry;
   optionally, adding at least one additional antibiotic agent to the slurry;
   disposing the slurry in a bead mold;
   allowing the slurry to set to form the antibiotic bead;
   collecting the antibiotic bead from the bead mold.
Embodiment 43. The method of embodiment 42, wherein the weight ratio of the aqueous solution and the dry mixture is at least 4:1 (e.g., at least 3:1).
Embodiment 44. The method of embodiment 42 or embodiment 43, comprising adding at least one additional antibiotic agent to the slurry.
Embodiment 45. A package comprising a plurality of the antibiotic beads of any of embodiments 1-16 having a size of from about 2 to about 8 millimeters, wherein the beads comprise one or more antibiotic agents dispersed within a magnesium phosphate and/or a magnesium sulfate carrier.
Embodiment 46. The package of embodiment 45, wherein the package contains from 2 to 1600 antibiotic beads.
Embodiment 47. The package of embodiment 45 or embodiment 46, wherein the package contains a plurality of first beads comprising a first antibiotic agent and a plurality of second beads comprising a second antibiotic agent, wherein the first antibiotic agent is different than the second antibiotic agent.
Embodiment 48. The package of any of embodiments 45-48, wherein the package contains a plurality of first beads comprising an antibiotic agent at a first concentration and a plurality of second beads comprising an antibiotic agent at a second concentration, wherein the second concentration is greater than the first concentration.
Embodiment 49. A method for treating an infection, the method comprising:
   disposing the bead of any of embodiments 1-41 within a wound site of a patient.
Embodiment 50. The method of embodiment 49, wherein the bead is allowed to remain within the wound site for a time period in the range of about 5 to 90 days or more (e.g., of about 5 to 70 days, about 5 to 50 day, or about 5 to 20 days).
Embodiment 51. The method of embodiment 49, wherein the bead is allowed to remain within the wound site for a time period in the range of about 5 to 10 days.
Embodiment 52. The method of any of embodiments 49-51, wherein the wound site includes a void in which infectious bone has been surgically removed, and wherein the bead is disposed within the void.
Embodiment 53. A method for treating an infection, the method comprising:
   disposing a plurality of beads within a wound site of a patient that have a size of from about 2 to about 8 millimeters, wherein the beads comprise one or more antibiotic agents dispersed within a magnesium phosphate and/or a magnesium sulfate carrier.
Embodiment 54. The method of embodiment 53, wherein the beads are allowed to remain within the wound site for a time period in the range of about 10 to 20 days.
Embodiment 55. A method for treating an infection, the method comprising:
   disposing a plurality of beads within a wound site of a patient that have a size of from about 2 to about 8 millimeters, wherein the beads comprise one or more antibiotic agents dispersed within a magnesium phosphate and/or a magnesium sulfate carrier.
Embodiment 56. The method of embodiment 55, wherein the beads are allowed to remain within the wound site for a time period in the range of about 20 to 90 days or more.
Embodiment 57. A method for treating an infection in soft tissue, the method comprising: disposing a plurality of beads within a wound site of a patient that have a size of from about 2 to about 8 millimeters, wherein the beads comprise one or more antibiotic agents dispersed within a magnesium phosphate and/or a magnesium sulfate carrier; and allowing soft tissue to form.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims, along with the full scope of equivalents to which such claims are entitled. It is also to be understood that the terminology used herein is for the purpose of describing particular embodiments only, and is not intended to be limiting.

Because many modifications, variations, and changes in detail can be made to the described example, it is intended that all matters in the preceding description and shown in the accompanying figures be interpreted as illustrative and not in a limiting sense. Further, it is intended to be understood that the following clauses (and any combination of the clauses) further describe aspects of the present description.

What is claimed is:

1. An antibiotic bead having a size of from about 2 to about 8 millimeters, wherein the antibiotic bead comprises:
one or more antibiotic agents dispersed within a magnesium phosphate and/or a magnesium sulfate carrier.

2. The antibiotic bead of claim 1, wherein the antibiotic bead comprises a magnesium phosphate carrier.

3. The antibiotic bead of claim 1, wherein the antibiotic bead comprises a magnesium sulfate carrier.

4. The antibiotic bead of claim 1, wherein the magnesium phosphate and/or magnesium sulfate carrier is prepared by mixing a dry mixture with an aqueous solution, wherein the dry mixture comprises MgO, magnesium sulfate, monobasic potassium phosphate, monobasic sodium phosphate, and calcium phosphate.

5. The antibiotic bead of claim 4, wherein a weight percent ratio of monobasic potassium phosphate to MgO is between about 3:1 and 1:1.

6. The antibiotic bead of claim 1, wherein the antibiotic bead has a generally circular cross-sectional shape, has a generally hexagon cross-sectional shape, or has a generally square cross-sectional shape.

7. The antibiotic bead of claim 1, wherein the antibiotic bead has an average normalized closed pore volume (i.e., PV/SV) in the range of 0.2 to 0.6%.

8. The antibiotic bead of claim 1, wherein the antibiotic bead has an average normalized closed pore volume (i.e., PV/SV) at least 1.2 times larger than a comparable bead not including the antibiotic agent.

9. The antibiotic bead of claim 1, wherein the antibiotic bead has an average normalized closed pore volume that increases when contacted with an aqueous solution in the range of 1.5%-4.5%.

10. The antibiotic bead of claim 1, wherein the antibiotic bead comprises more antibiotic agent than an antibiotic agent of an otherwise identical bead comprising a calcium compound instead of a magnesium phosphate and/or a magnesium sulfate carrier after contact with an aqueous solution for a time period of at least 5 days.

11. The antibiotic bead of claim 1, wherein the antibiotic bead has an elution rate that is slower than an elution rate of an otherwise identical bead comprising a calcium compound instead of a magnesium phosphate and/or a magnesium sulfate carrier over a time period of 9 days of contact with an aqueous solution.

12. The antibiotic bead of claim 1, wherein at least one of the one or more antibiotic agents selected is an aminoglycoside antibiotic.

13. The antibiotic bead of claim 1, wherein at least one of the one or more antibiotic agents selected is an glycopeptide antibiotic.

14. The antibiotic bead of claim 1, wherein the one or more antibiotic agents is selected from vancomycin, gentamycin, tobramycin, doxycycline, or minocycline.

15. The antibiotic bead of claim 1 comprising two or more antibiotic agents.

16. The antibiotic bead of claim 15, wherein the two or more antibiotic agents are selected from vancomycin, gentamycin, tobramycin, doxycycline, or minocycline.

17. The antibiotic bead of claim 1, wherein the antibiotic bead further comprises a release modifier, a radiocontrast agent, a colorizing agent, an antimicrobial agent and/or preservative.

18. The antibiotic bead of claim 1 having a calcium sulfate content of no more than 5 wt %, based on the total weight of the bead.

19. The antibiotic bead of claim 1 comprising substantially no calcium sulfate.

20. A method of making the antibiotic bead of claim 1, the method comprising providing an aqueous solution comprising one or more antibiotic agents;
providing a dry mixture comprising MgO, magnesium sulfate, monobasic potassium phosphate, monobasic sodium phosphate, and calcium phosphate, wherein a weight percent ratio of monobasic potassium phosphate to MgO is between about 3:1 and 1:1;
mixing the aqueous solution and the dry mixture in a weight ratio of at least 5:1 to from a slurry;
optionally, adding at least one additional antibiotic agent to the slurry;
disposing the slurry in a bead mold;
allowing the slurry to set to form the antibiotic bead;
collecting the antibiotic bead from the bead mold.

21. A package comprising a plurality of the antibiotic beads of claim 1 having a size of from about 2 to about 8 millimeters, wherein the beads comprise one or more antibiotic agents dispersed within a magnesium phosphate and/or a magnesium sulfate carrier.

22. A method for treating an infection, the method comprising:
disposing the bead of claim 1 within a wound site of a patient.

23. A method for treating an infection in soft tissue, the method comprising:
disposing a plurality of beads within a wound site of a patient that have a size of from about 2 to about 8 millimeters, wherein the beads comprise one or more antibiotic agents dispersed within a magnesium phosphate and/or a magnesium sulfate carrier; and
allowing soft tissue to form.
